(19) United States
(12) Patent Application Publication
(10) Pub. No.: US 2010/0251394 A1
(43) Pub. Date: Sep. 30, 2010

(54) TREATMENT AND PREVENTION OF ISCHEMIC BRAIN INJURY

(75) Inventor: Sylvain Doré, Baltimore, MD (US)

Correspondence Address: BANNER & WITCOFF, LTD., 1100 13th STREET, N.W., SUITE 1200, WASHINGTON, DC, 20005-4051, US

(73) Assignee: THE JOHNS HOPKINS UNIVERSITY, Baltimore, MD (US)

(21) Appl. No.: 12/678,028

(22) PCT Filed: Sep. 22, 2008
(86) PCT No.: PCT/US08/77185

§ 371 Date: May 20, 2010

Related U.S. Application Data
(60) Provisional application No. 60/994,645, filed on Sep. 20, 2007.

(51) Int. Cl. A61K 49/00

(52) U.S. Cl. 800/9

(57) ABSTRACT

The invention provides methods of identifying agents for treating and preventing ischemic brain injury.

This application claims the benefit of and incorporates by reference Ser. No. 60/994,645 filed Sep. 20, 2007.

This invention was made using funds from NIH grants NS046400 and AG022971. The government therefore retains certain rights in the invention.

### FIELD OF THE INVENTION

The invention relates to methods of identifying agents for treating and preventing ischemic brain injury.

### BACKGROUND OF THE INVENTION

Cerebral ischemia, also referred to as stroke, is an acute neurological injury resulting from occlusion or hemorrhage of blood vessels supplying oxygen and important nutrients to the brain (del Zoppo & Koziol, 2007; Durukan & Tatlisumak, 2007). An ischemic cascade is initiated that causes brain cells to die or to be seriously damaged and causes neurological impairments that reflect brain damage severity.

Mechanisms involved in development and progression of cerebral ischemia are complex and include excitotoxicity, ionic imbalance, oxidative/nitrosative stress, and inflammation (Finley et al., 2006; Weinberger, 2006). Inflammation triggers the activation of phospholipase A₂ and the release of arachidonic acid from membrane phospholipids (Khanapure et al., 2007). The arachidonic acid is ultimately converted by cyclooxygenase (COX) and lipoxygenase pathways to prostaglandins (PGs), thromboxane A₂ (TXA₂), and leukotrienes (LTs), collectively termed eicosanoids. Both COX and lipoxygenase pathways have clinical importance in relation to several human disorders (Tomimoto et al., 2002).

Non-steroidal anti-inflammatory drugs (NSAIDs), the most popular medications used to treat pain, heart disease, fever, and inflammation, mainly target the COX pathway (Bishop-Bailey et al., 2006; Premkumar & Raisinghani, 2006). COX 1 and COX 2 catalyze the synthesis of PGH₂, the prevalent substrate for the production of prostanoids PGE₂, PGD₂, PGF_{2α}, prostacyclin (PG1₂), and TXA₂. Prostanoids in turn participate in inflammatory responses by binding with G protein-coupled receptors designated EP(1-4), FP, DP, IP, and TP, respectively (Doré, 2006).

PGD₂ is a lipid mediator that is highly abundant in the central nervous systems of rats, mice, and humans (Doniach, 1977; Hayaishi, 1991) and is involved in various pathophysiological events, such as regulation of the sleep/wake cycle, pain response, hypoxia, seizure, and inflammation (Eguchi et al., 1999; Hayaishi, 2002; Chen & Bazan, 2005; Taniguchi et al., 2007). PGD₂ is synthesized by hematopoietic prostaglandin D synthetase (H-PGDS) and lipocalin-type PGDS (L-PGDS) and mediates its function by binding to specific receptors known as DP1 and DP2 (Kabashima and Narumiya, 2003; Nagata and Hirai, 2003). In the brain, L-PGDS is expressed in leptomeninges, choroid plexus, and oligodendrocytes (Urade et al., 1993; Beuckmann et al., 2000; Mohri et al., 2006a; Urade & Mohri, 2006), whereas H-PGDS is expressed in ameboid and ramified forms of microglia (Mohri et al., 2003). L-PGDS is known to play important roles in spinal cord injury, multiple sclerosis, atherosclerosis, and hypertension (Hirawa et al., 2002; Miwa et al., 2004; Grill et al., 2006; Kagitani-Shimono et al., 2006). Recent reports suggest that focal cerebral ischemia induces H-PGDS in microglia and macrophages (Liu et al., 2007).

There is a need in the art for agents that can treat or prevent brain damage due to cerebral ischemia.

### SUMMARY OF THE INVENTION

One embodiment of the invention comprises contacting a lipocalin-type prostaglandin synthetase (L-PGDS) with a test compound; determining whether the test compound increases enzymatic activity of the L-PGDS; and determining whether the test compound prevents or decreases a symptom of ischemic brain injury in an animal model.

Another embodiment of the invention comprises contacting a mammalian cell which expresses L-PGDS with a test compound; determining whether the test compound increases expression of L-PGDS; and determining whether the test compound prevents or decreases a symptom of ischemic brain injury in an animal model.

### BRIEF DESCRIPTION OF THE DRAWINGS

FIGS. 1A-C. Genetic deletion of L-PGDS does not significantly affect cerebral arterial vasculature in mice. Photographs showing the cerebral arterial vasculature of wild-type (WT; FIG. 1A) and L-PGDS^{-/-} (FIG. 1B) mice. FIG. 1C, macroscopic analysis of cerebral arterial vasculature did not reveal differences in the circle of Willis or major cerebral arteries between L-PGDS^{-/-} and WT mice (n=3/group).

FIGS. 2A-C. Absence of L-PGDS does not affect the monitored physiological parameters. Relative cerebral blood flow (CBF, FIG. 2A), core body temperature (FIG. 2B), and mean arterial blood pressure (MABP, FIG. 2C) were recorded at baseline (ctrl), at induction of ischemia (immed), and at 15-min intervals during ischemia and 1 h of reperfusion. Change in CBF was recorded as a percent of baseline.

FIGS. 3A-C. Deletion of L-PGDS enhances ischemic brain injury and neurological dysfunction after transient ischemia. FIG. 3A, photographs of infarcted brain slices from WT (left) and L-PGDS^{-/-} (right) mice. FIG. 3B, percent corrected hemispheric infarct volume was significantly larger in L-PGDS^{-/-} mice than in WT mice after 90 min of ischemia and 4 days of reperfusion. FIG. 3C, neurological scores assessed 4 days after ischemia were significantly higher in L-PGDS^{-/-} mice than in WT mice, indicating greater neurological dysfunction. *P<0.01 vs WT.

FIG. 4. Water content in ischemic and nonischemic hemispheres of WT (n=4) and L-PGDS^{-/-} (n=5) mice 4 days after transient middle cerebral artery occlusion. Values are expressed as means±SEM. *P<0.01.

FIGS. 5A-C. Deletion of L-PGDS enhances ischemic brain injury and neurological dysfunction after permanent distal middle cerebral artery occlusion. FIG. 5A, photographs of infarcted brain slices from WT (left) and L-PGDS^{-/-} (right) mice. FIG. 5B, percent corrected cortical infarct volume was significantly larger in L-PGDS^{-/-} mice than in WT mice after 7 days. FIG. 5C, neurological scores assessed 7 days after ischemia were significantly higher in L-PGDS^{-/-} mice than in WT mice, indicating greater neurological dysfunction. *P<0.01 vs WT.

### DETAILED DESCRIPTION

The invention provides methods for identifying agents which increase expression and/or activity of L-PGDS for use in treating and preventing ischemic brain injury by administering agents. The methods can be carried out in vivo or in vitro.

In one embodiment, test compounds are screened for the ability to increase enzymatic activity of L-PGDS. L-PGDS protein is contacted with a test compound, either in vitro or in vivo, and the activity of L-PGDS is determined. The level of enzymatic activity of L-PGDS in the presence of the test compound is compared to the level of enzymatic activity of L-PGDS in the absence of the test compound. Test compounds that increase L-PGDS enzymatic activity can be identified based on this comparison.

In another embodiment, test compounds are screened for the ability to affect L-PGDS gene expression such that increased levels of L-PGDS protein are produced. A cell which naturally expresses L-PGDS (e.g., leptomeningeal cells, choroid plexus cells, or oligodendrocytes) is contacted with a test compound, and the expression of L-PGDS is determined. The level of expression of L-PGDS in the presence of the test compound is compared to the level of expression of L-PGDS in the absence of the test compound. Test compounds that increase L-PGDS gene expression can be identified based on this comparison.

The level of L-PGDS enzymatic activity or gene expression can be determined by methods well known in the art, as described below. Either qualitative or quantitative methods can be used.

### Test Compounds

According to the present invention, test compounds are tested for the ability to increase L-PGDS enzymatic activity and/or expression of L-PGDS protein. Test compounds for use in methods of the invention can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art.

Test compounds can be obtained using any of the numerous combinatorial library methods known in the art, including but not limited to, biological libraries, spatially addressable parallel solid phase or solution phase libraries, synthetic library methods requiring deconvolution, the “one bead one compound” library method, and synthetic library methods using affinity chromatography selection. Methods for the synthesis of molecular libraries are well known in the art (see, for example, DeWitt et al., Proc. Natl. Acad. Sci. U.S.A. 90, 6909, 1993; Erb et al., Proc. Natl. Acad. Sci. U.S.A. 91, 11422, 1994; Zuckermann et al., J. Med. Chem. 37, 2678, 1994; Cho et al., Science 261, 1303, 1993; Carell et al., Angew. Chem. Int. Ed. Engl. 33, 2059, 1994; Carell et al., Angew. Chem. Int. Ed. Engl. 33, 2061; Gallop et al., J. Med. Chem. 37, 1233, 1994). Libraries of compounds can be presented in solution (see, e.g., Houghten, BioTechniques 13, 412-21, 1992), or on beads (Lam, Nature 354, 82-84, 1991), chips (Fodor, Nature 364, 555-56, 1993), bacteria or spores (Ladner, U.S. Pat. No. 5,223,409), plasmids (Cull et al., Proc. Natl. Acad. Sci. U.S.A. 89, 1865-869, 1992), or phage (Scott & Smith, Science 249, 386-90, 1990; Devlin, Science 249, 404-06, 1990); Cwirla et al., Proc. Natl. Acad. Sci. 97, 6378-82, 1990; Felici, J. Mol. Biol. 222, 301-10, 1991; and Ladner, U.S. Pat. No. 5,223,409).

### L-PGDS Protein

L-PGDS protein for use in screening methods of the invention can be produced recombinantly, purified from natural sources, or synthesized chemically. The amino acid sequence of L-PGDS protein (also known as “beta trace protein”) is known. See Kuruvilla et al., Brain Research 56, 337-40, 1991; Zahn et al., Neuroscience Letters 154, 93-95, 1993; Hoffmann et al., J. Neurochem. 61, 451-56, 1993; Watanabe et al., Biochem. Biophys. Res. Communication 203, 1110-16, 1994. L-PGDS can be produced using routine expression methods, for example in E. coli or CHO cells comprising an expression vector encoding L-PGDS. Nagata et al., Proc. Natl. Acad. Sci. USA 88, 4020-24, 1991 discloses a cDNA which can be used to encode L-PGDS, although any coding sequence which encodes the protein can be used. Using well-known recombinant DNA methods, a recombinant DNA containing the cDNA for L-PGDS can be constructed and transformed into a host cell, which can then cultured to produce the enzyme.

L-PGDS can be purified from the culture by conventional means. L-PGDS also can be purified from natural sources (e.g., leptomeninges, choroid plexus, oligodendrocytes) using methods well known in the art. Such methods include, but are not limited to, size exclusion chromatography, ammonium sulfate fractionation, ion exchange chromatography, affinity chromatography, and preparative gel electrophoresis. Purification of L-PGDS is described, e.g., in Ragolin et al., Prostaglandins Other Lipid Mediat. 83, 25-32, 2007.

L-PGDS can be produced using chemical methods to synthesize its amino acid sequence, such as by direct peptide synthesis using solid-phase techniques (Merrifield, J. Am. Chem. Soc. 85, 2149-2154, 1963; Roberge et al., Science 269, 202-204, 1995), which is incorporated herein by reference in its entirety. Protein synthesis can be performed using manual techniques or by automation. Automated synthesis can be achieved, for example, using Applied Biosystems 431A Peptide Synthesizer (Perkin Elmer). Optionally, fragments of L-PGDS can be separately synthesized and combined using chemical methods to produce a full-length molecule. See WO 01/98340.

### Methods of Measuring L-PGDS Levels

Any method can be used to measure L-PGDS levels in response to a test compound.

Examples include immunological assay methods, using a monoclonal antibody or a polyclonal antibody specific to L-PGDS, such as enzyme immunoassay methods, radioimmunoassay methods, latex agglutination assay methods, fluorescence immunoassay methods, “sandwich” immunoassays, immunoradiometric assays, gel diffusion precipitation reactions, immunodiffusion assays, in situ immunoassays (for example, using colloidal gold, enzyme or radioisotope labels), Western blots, precipitation reactions, agglutination assays (e.g., gel agglutination assays or hemagglutination assays), complement fixation assays, immunofluorescence assays, protein A assays, and immunoelectrophoresis assays. US 2004/0038314 discloses monoclonal antibodies which can be used to detect L-PGDS.

WO97/16461 discloses a sandwich ELISA method for detecting L-PGDS. Other methods for immunohistochemical detection of L-PGDS (e.g., Western blotting, ELISA) are described in US 2004/0038314 and US 2007/0020609. Detection of L-PGDS by Western blotting also is described in Joo et al., J. Immunol. 179, 2565-75, 2007.

In other embodiments, L-PGDS levels can be detected using a chromatographic method in which the presence of L-PGDS produces a detectable color change. In other embodiments an enzymatic method in which the presence of L-PGDS initiates detectable enzymatic activation is used. In further embodiments the presence of L-PGDS in a sample is detected by atomic absorption or atomic emission spectroscopy. See US 2008/0196864.

### L-PGDS Enzyme Activity

Methods of measuring L-PGDS enzyme activity are known in the art and are disclosed, for example, in Inui et al., Biochem. Biophys. Res. Commun. 266, 641-46, 1999; Ragolin et al., Am. J. Physiol. Cell. Physiol. 284, C119-C125, 2002; Ragolin et al., Prostaglandins Other Lipid Mediat. 83, 25-32, 2007; and Li et al., Neurol. 70, 1753-62, 2008.

### High Throughput Screening

Screening methods of the invention can be used in high through-put screening formats. Using high throughput screening, many discrete compounds can be tested in parallel so that large numbers of test compounds can be quickly screened. The most widely established techniques utilize 96-well microtiter plates, however 384- or 1536-plates also can be used. As is known in the art, a variety of instruments, materials, pipettors, robotics, plate washers, and plate readers are commercially available.

### Animal Models

After a test compound is identified as able to increase L-PGDS protein levels or enzymatic activity, the test compound is administered to an animal model to determine whether it can reduce or prevent a symptom of brain ischemia. Animal models of brain ischemia are well known in the art. A useful model is described in Example 1. Symptoms that can be assessed include infarct volume, limb weakness, torso turning to the ipsilateral side of the lesion, circling to the affected side, ability to bear weight on the affected side, degree of locomotor activity or barrel rolling, mean arterial blood pressure, and blood chemistry (e.g., pH, PaCO₂, PaO₂). See Example 1. A test compound preferably reduces one or more of these symptoms by 10, 20, 30, 40, 50, 60, 70, 80, 90, or 100%.

All patents, patent applications, and references cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLES

Prostaglandin (PG) D₂ is produced as a metabolite of arachidonic acid via the cyclooxygenase pathway in the brain. Synthesized by hematopoietic PGD₂ synthetases (H-PGDS) or lipocalin-type PGDS (L-PGDS), PGD₂ is produced by various organs and cell types and then would bind preferentially to DP1 or DP2 receptors. Relatively little is known about the role of L-PGDS in cerebral ischemia. In the examples below, outcomes in L-PGDS knockout and wildtype (WT) mice in a transient cerebral focal ischemia model were compared. Briefly, the middle cerebral artery (MCA) of mice was occluded with a 7-0 nylon monofilament for 90 min. The percent corrected infarct volume and brain edema were determined after 4 days of reperfusion. In a separate cohort of animals, physiological parameters (mean arterial blood pressure, pH, PaCO₂, and PaO₂) were measured before and during MCA occlusion and for 60 min of reperfusion. L-PGDS knockout mice had significantly (p<0.01) higher percent corrected infarct volume and brain edema than did WT mice. There were no significant differences in physiological outcomes. Also, for the permanent distal MCAO, the mice under halothane anesthesia, a 1.0-cm vertical skin incision was made between the right eye and ear. The temporal muscle was moved, and the temporal bone exposed and 2.0-mm burr hole was made just over the MCA, visible through the temporal bone. The main trunk of the distal part of the MCA was directly occluded with a bipolar coagulator, and complete interruption of blood flow at the occlusion site was confirmed by severance of the occlusion site of the MCA. Animals not circling toward the paretic side after the onset of ischemia and those that developed subarachnoid hemorrhage were eliminated from the study. A successful occlusion was also confirmed by placing the laser-Doppler probe above the temporal ridge to establish that blood flow into the region was terminated. After 7 days, the mice were euthanized and the brains harvested. To determine the neurological deficits caused by this model, a 28-point score pattern was used. Seven days after the pMCAO procedure, an experimenter blinded to genotype scored all mice for neurological deficits. The tests included body symmetry, gait, climbing, circling behavior, front limb symmetry, compulsory circling, and whisker response. Each test was graded from 0 to 4, establishing a maximum deficit score of 28. These findings suggest that L-PGDS is important for the protection of brain against cerebral ischemia.

### Example 1

### Materials and Methods

This study was performed in accordance with the NIH guidelines for the use of experimental animals; protocols were approved by the Johns Hopkins Animal Care and Use Committee. C57BL/6 WT and L-PGDS^{-/-} mice were bred and genotyped for this study.

Anatomical examination of cerebrovasculature. Adult male WT and L-PGDS^{-/-} mice (20-25 g; n=3/group) were deeply anesthetized with halothane before being perfused by cardiac puncture with saline followed by black latex paint. Then the brains were carefully harvested and immersed in 10% formalin for 24 h before examination. The vessel diameters were evaluated with Metavue software (Meta Imaging Series Software, Downingtown, Pa., USA).

Transient ischemia protocol and assessment of neurological score. Transient focal cerebral ischemia was induced by tMCAO with an intraluminal filament technique as described (Ahmad et al., 2006). Briefly, adult male mice (20-28 g) were placed under halothane anesthesia. Body temperature was maintained at 37.0±0.5° C. with a heating pad. Relative cerebral blood flow (CBF) was monitored by laser-Doppler flowmetry (Moor instruments, Devon, England) over the parietal cortex supplied by the MCA. Occlusion of the MCA was accomplished with a 7-0 Ethilon nylon monofilament (Ethicon, Somerville, N.J., USA) coated with flexible silicone and confirmed by a decrease in CBF. During the 90-min occlusion, anesthesia was discontinued, and the animals were transferred to a humidity- and temperature-controlled chamber; animal behavior was also monitored for the entire time period to further confirm the occlusion. Then the mice were re-anesthetized, and the filament was withdrawn. The mice were returned to the chamber for approximately 6 h before being returned to their home cages. Neurological function was measured in each mouse on day 4 after reperfusion according to the following graded scoring system: 0=no deficit; 1=forelimb weakness and torso turning to the ipsilateral side when held by tail; 2=circling to affected side; 3=unable to bear weight on affected side; and 4=no spontaneous locomotor activity or barrel rolling.

Measurement of body temperature, blood gases, and mean arterial blood pressure. In a separate cohort of animals (n=5/genotype), the femoral artery was cannulated for measurement of arterial blood gases and mean arterial blood pressure (MABP) at baseline and at 15-min intervals for 90 min of ischemia and 60 min of reperfusion. Body temperature was determined with a rectal probe at the same time points.

Brain Water Content. In another cohort of mice (n=5/genotype), brain water content was measured by the wet/dry weight method. Mice were deeply anesthetized with halothane and decapitated to remove their brains. Samples were taken from ischemic and nonischemic hemispheres. The brains were weighed wet, oven dried at 100° C. for 48 h, and then reweighed. Brain water content (%) was calculated as (wet weight—dry weight)/wet weight×100.

Permanent distal MCAO and assessment of neurological score. For the pMCAO protocol, with the mice under halothane anesthesia, a 1.0-cm vertical skin incision was made between the right eye and ear. The temporal muscle was moved, and the temporal bone exposed. Under a surgical microscope, a 2.0-mm burr hole was made just over the MCA, visible through the temporal bone. The main trunk of the distal part of the MCA was directly occluded with a bipolar coagulator, and complete interruption of blood flow at the occlusion site was confirmed by severance of the occlusion site of the MCA. Core body temperature was maintained between 36.5 and 37.5° C. during and after the procedures. Animals not circling toward the paretic side after the onset of ischemia and those that developed subarachnoid hemorrhage were eliminated from the study. A successful occlusion was also confirmed by placing the laser-Doppler probe above the temporal ridge to establish that blood flow into the region was terminated. After 7 days, the mice were euthanized and the brains harvested. To determine the neurological deficits caused by this model, a robust 28-point score pattern was used (Wang et al., 2006). Seven days after the pMCAO procedure, an experimenter blinded to genotype scored all mice for neurological deficits. The tests included body symmetry, gait, climbing, circling behavior, front limb symmetry, compulsory circling, and whisker response. Each test was graded from 0 to 4, establishing a maximum deficit score of 28. Immediately after the testing, the mice were sacrificed for infarct volume analysis.

Quantification of infarct volume in both ischemic stroke models. After neurological assessment, the mice were anesthetized deeply, and the brains were harvested and sliced coronally into five 2-mm thick sections, which were incubated with 1% 2, 3, 5-triphenyltetrazolium chloride (TTC) in saline for 30 min at 37° C. The area of brain infarct, identified by the lack of TTC staining, was measured on the rostral and caudal surfaces of each slice and numerically integrated across the thickness of the slice to obtain an estimate of infarct volume (Sigma Scan Pro, Systat, Port Richmond, Calif., USA). Volumes of all five slices were summed to calculate total infarct volume over the entire hemisphere and expressed as a percentage of the volume of the contralateral hemisphere. Infarct volume was corrected for swelling by comparing the volumes in the ipsilateral and contralateral hemispheres. The corrected volume of the infarcted hemisphere was calculated as volume of contralateral hemisphere—(volume of ipsilateral hemisphere—volume of infarct) (Doré et al., 2003).

### Example 2

### Effect of L-PGDS on Transient Focal Ischemia-Induced Brain Injury

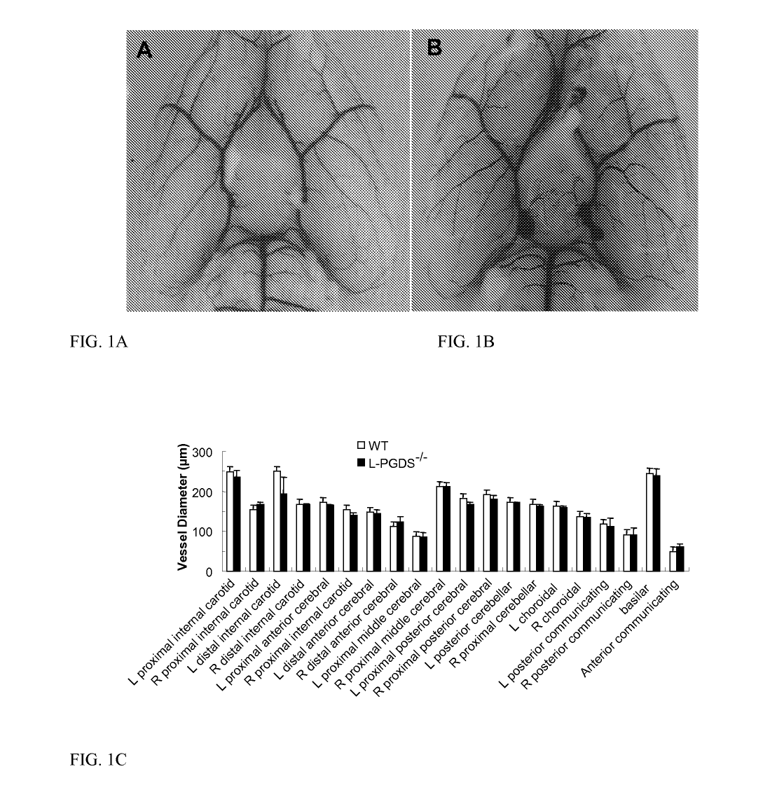

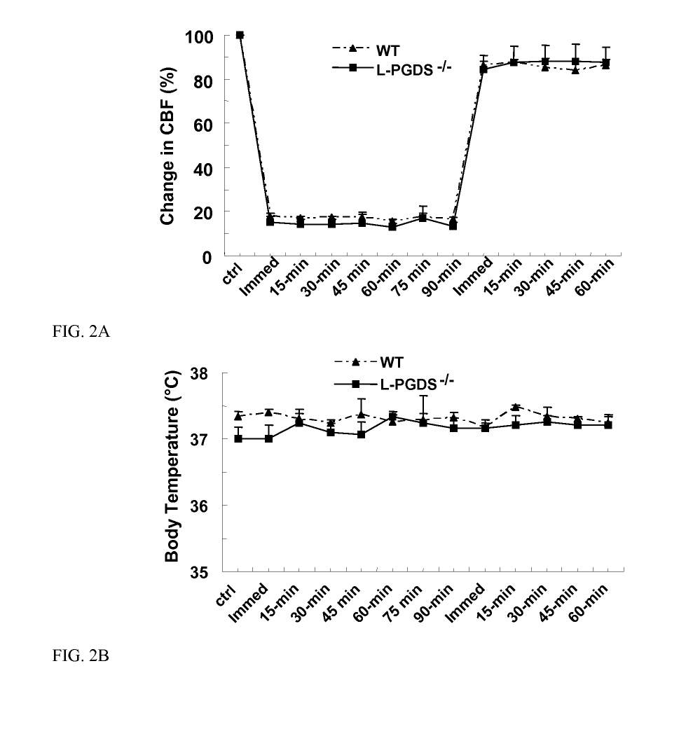

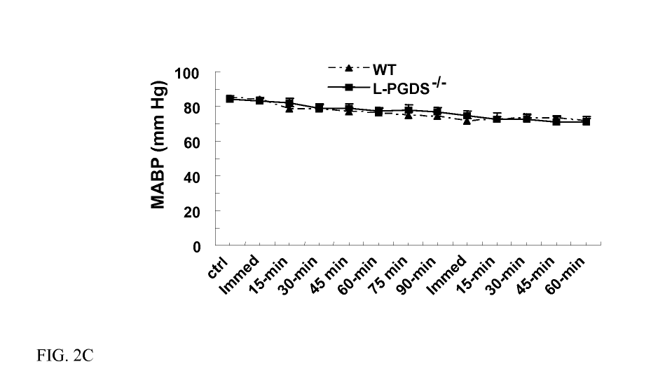

This example describes the effect of L-PGDS on transient focal ischemia-induced brain injury. Pathophysiological outcomes after MCAO in WT and L-PGDS^{-/-} mice were compared. Examination of the large cerebral vessels in WT and L-PGDS^{-/-} mice revealed no significant differences between the two groups. Similarly, there were no significant differences in blood vessel diameters, MABP or blood gases (pH, PaCO₂, and PaO₂) between WT and L-PGDS-1-mice (FIGS. 1-2; Table 1).

TABLE 1
Effect of MCAO on physiological parameters in WT and L-PGDS^{-/-} mice

| | wild-type mice | | | L-PGDS^{-/-} mice | | |
|---|---|---|---|---|---|---|
| Parameter | baseline | 1 h MCAO | 1 h reperfusion | baseline | 1 h MCAO | 1 h reperfusion |
| pH | 7.37 ± 0.02 | 7.34 ± 0.01 | 7.35 ± 0.01 | 7.34 ± 0.01 | 7.34 ± 0.01 | 7.33 ± 0.01 |
| PaCO₂ | 38.7 ± 1.2 | 40.3 ± 1.1 | 39.7 ± 1.5 | 39.1 ± 1.1 | 39.4 ± 1.0 | 39.5 ± 1.0 |
| PaO₂ | 108 ± 2 | 128 ± 4 | 112 ± 5 | 111 ± 5 | 127 ± 7 | 114 ± 3 |

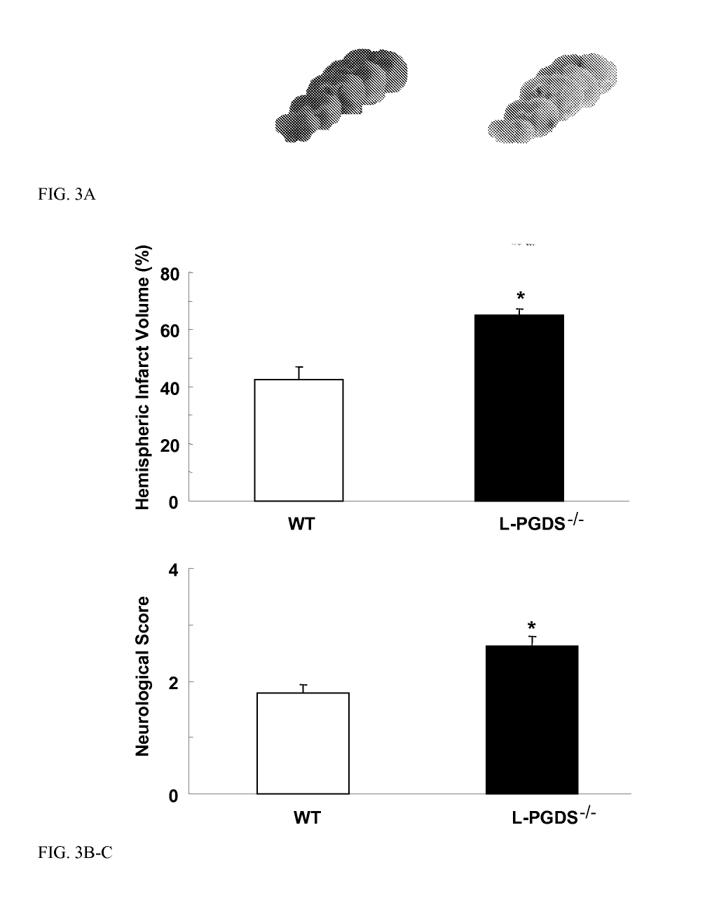

However, the mean infarct size of mice lacking L-PGDS was significantly greater (p<0.01) than that of WT mice after 90 min of MCAO and 4 days of reperfusion (FIG. 3). Furthermore neurological scores of the L-PGDS^{-/-} mice were significantly higher than those of the WT mice (p<0.01; FIG. 3), indicating greater dysfunction.

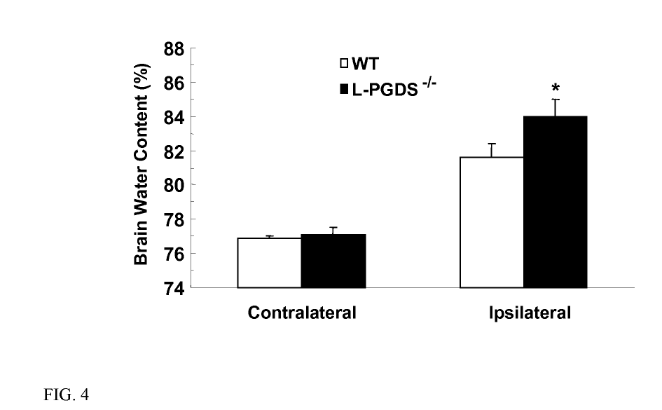

The water content in each hemisphere of the WT and L-PGDS^{-/-} mice is given in FIG. 4. When the ipsilateral hemispheres were compared, the water content of the LPGDS^{-/-} mice was significantly higher than that of the WT mice. However, the water content of the respective contralateral hemispheres did not differ substantially between the two genotypes.

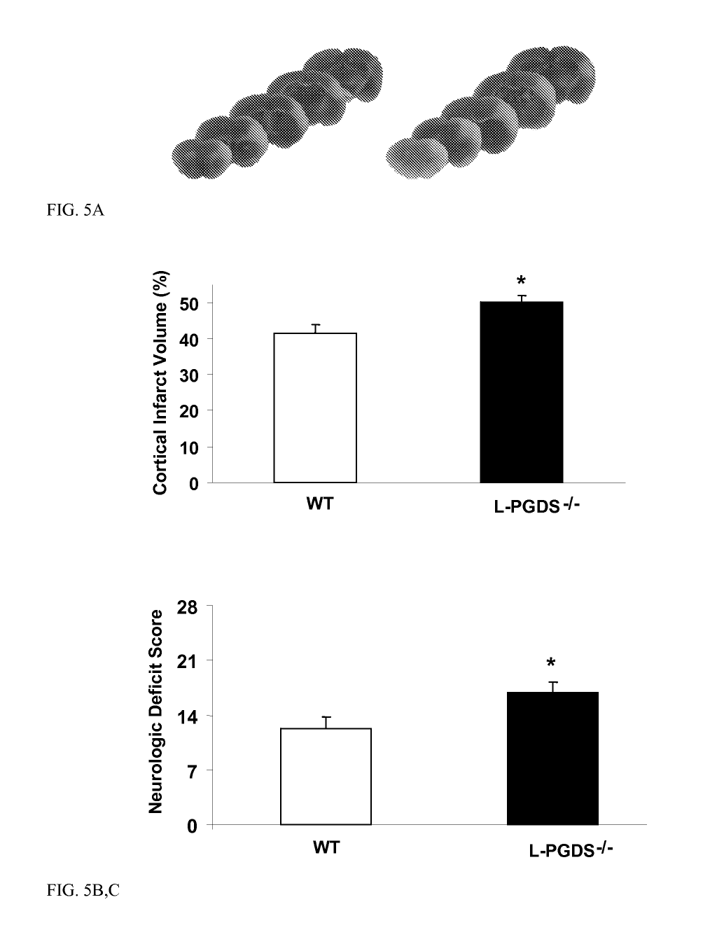

Furthermore, after 7 days of permanent occlusion, L-PGDS^{-/-} mice had significantly larger infarct size (49.7±1.7%; P<0.02) than did their counterpart WT mice (FIG. 5). And, pMCAO produced significantly higher neurological deficit scores in L-PGDS^{-/-} mice (16.6±1.3%; P<0.02) than in WT mice (FIG. 5C).

Thus, neurological dysfunction, percent corrected infarct volume, and water content of the ipsilateral hemisphere were significantly higher in the knockout mice than in the WT mice. However, MABP, blood chemistry, and water content of the contralateral hemisphere were not significantly different between the two groups of mice. These results suggest that absence of L-PGDS exacerbates the ischemic brain injury without affecting the physiological parameters.

PGD₂ is considered to be the most abundant PG present in brain. It is well known that inflammation increases PGD₂ synthesis in the central nervous system (Hetu & Riendeau, 2005; Mohri et al., 2006b). Published reports indicate that two PGDS enzymes (H-PGDS and L-PGDS) regulate the synthesis of PGD₂, which mainly acts through two different G-protein-coupled receptors, namely DP1 (Bole et al., 1995) and CRTH2 (chemo attractant homologous receptor expressed on TH2 cells), also known as DP2 (Hirai et al., 2001). The DP1 receptor plays a protective role in ischemia/reperfusion injury (Saleem, 2007). However, It is important to determine the role of brain L-PGDS during ischemic stroke because it may constitute a mechanism by which brain PGD₂ biosynthesis can be regulated independent of other PGs. It has been suggested that during the course of inflammation, a shift occurs between initial dominance of PGE₂ towards PGD₂ biosynthesis during later stages of inflammation, a response that may contribute to resolution of inflammation (Gilroy et al., 2003). The proposed function of PGD₂ in the resolution phase of inflammation makes this compound a unique member of the PG family.

Recent studies have indicated that variation in the cerebrovasculature of animals affects stroke outcome in global and focal cerebral ischemia. Here, we compared the cerebrovascular anatomy of WT and L-PGDS^{-/-} mice and found no significance difference between the two groups, signifying that differences in outcome did not stem from differences in structure. Further, it was important to evaluate the effect of L-PGDS absence on physiological parameters such as CBF, MABP, body temperature, and blood gases (pH, PaCO₂ and PaO₂) because the maintenance of physiological parameters reduces secondary disease or conditions that develop in the course of a primary disease. We chose to measure the CBF by laser Doppler flowmetry rather than by the standard method of ¹⁴C-iodoantipyrine because we needed to assess the temporal changes in blood flow in real time, a measurement that cannot be made with ¹⁴C-iodoantipyrine. Moreover, the laser Doppler method has become widely accepted in recent years.

The transient focal ischemia model produces large infarcts that significantly affect the striatum, extend to the cortical region, and often result in high mortality rates, particularly in certain knockout mice. Therefore, it is sometimes difficult to study long-term or delayed ischemic responses with this model. The pMCAO model used here provides an advantage because the damage is limited to the distal cortical region of the MCA territory, and the mice can be evaluated at later time points. Using the pMCAO model, we were able to study the role of L-PGDS in shaping the distal cortical damage of mice following permanent stroke and to evaluate neurological deficits 7 days after ischemic insult. The resulting data support the neuroprotective role of L-PGDS in stroke, as observed with the tMCAO model.

PGD₂ plays an important role in induction of brain edema (Taniguchi, H et al, 2007; Asano et al, 1985; Pappius et al., 1983). The physiological and molecular mechanisms of brain edema formation after ischemia/reperfusion-induced brain injury are complex (Xiao F et al., 2002). The pathobiology of ischemia-elicited cerebral edema includes a cytotoxic component (secondary to post ischemic failure of the Na⁺/K⁺ ATPase and other ATP-dependent transporters) and a vasogenic component (secondary to breakdown of the blood brain barrier, with leakage of plasma proteins into extra cellular space), although several other mechanisms, such as disruption of Ca²⁺ signaling, inflammatory mediators, neurohumoral responses, vascular endothelial growth factor, and upregulation of water channels, are now strongly implicated in ischemia-induced edema (Hosomi et al., 2005; Gerriets et al., 2004). In general, the distinction between cytotoxic edema and vasogenic edema is that the latter needs blood flow to cause swelling. The results presented in Example 2 indicate that absence of L-PGDS increases water content of the brain in the ipsilateral hemisphere but has no significant effect on the contralateral hemisphere.

The results of Example 2 suggest that genetic deletion of L-PGDS exacerbates ischemia/reperfusion brain injury without effecting the physiological parameters, as confirmed by measuring the physiological and histological parameters in WT and L-PGDS^{-/-} mice. In addition, loss of L-PGDS led to significantly more cortical damage than that observed in WT mice following distal pMCAO. In conclusion, the results of Example 2 provide evidence for the importance of L-PGDS following transient and permanent brain ischemic injury.

### REFERENCES

Ahmad et al. (2006b) 1-HydroxyPGE, reduces infarction volume in mouse transient cerebral ischemia. Eur J Neurosci 23:35-42.
Beuckmann et al. (2000) Cellular localization of lipocalin-type prostaglandin D synthase (beta-trace) in the central nervous system of the adult rat. J Comp Neurol, 428, 62-78.
Bishop-Bailey et al. (2006) COX-2 in cardiovascular disease. Arterioscler Thromb Vasc Biol, 26, 956-958.
Boie et al. (1995) Molecular cloning and characterization of the human prostanoid DP receptor. J. Biol. Chem., 270, 18910-18916.
Chen & Bazan (2005) Lipid signaling: sleep, synaptic plasticity, and neuroprotection. Prostaglandins Other Lipid Mediat., 77, 65-76.
del Zoppo & Koziol (2007) Recanalization and stroke outcome. Circulation, 115, 2602-2605. Doniach (1977) Histopathology of the anterior pituitary. Clin Endocrinol Metab, 6, 21-52.
Doré et al. (2003) Neuronal overexpression of cyclooxygenase-2 increases cerebral infarction. Ann Neurol., 54, 155-162.
Durukan & Tatlisumak (2007) Acute ischemic stroke: Overview of major experimental rodent models, path ophysiology, and therapy of focal cerebral ischemia. Pharmacol. Biochem. Behav., 87, 179-197.
Eguchi et al. (1999) Lack of tactile pain (allodynia) in lipocalin-type prostaglandin D synthase-deficient mice. Proc. Natl. Acad. Sci. U.S.A., 96, 726-730.
Finley Caulfield & Wijman (2006) Critical care of acute ischemic stroke. Crit. Care. Clin., 22, 581-606; abstract vii.
Gilroy et al. (2003) Inducible cyclooxygenase-derived 15-deoxy(Delta)12-14PGJ2 brings about acute inflammatory resolution in rat pleurisy by inducing neutrophil and macrophage apoptosis. Faseb J., 17, 2269-2271.
Grill et al. (2006) Systemic inflammation induces COX-2 mediated prostaglandin D2 biosynthesis in mice spinal cord. Neuropharmacology, 50, 165-173.
Hayaishi (1991) Molecular mechanisms of sleep-wake regulation: roles of prostaglandins D2 and E2. Faseb J., 5, 2575-2581.
Hayaishi (2002) Molecular genetic studies on sleep-wake regulation, with special emphasis on the prostaglandin D(2) system. J Appl Physiol, 92, 863-868.
Hetu & Riendeau (2005) Cyclo-oxygenase-2 contributes to constitutive prostanoid production in rat kidney and brain. Biochem. J., 391, 561-566.
Hirai et al. (2001) Prostaglandin D2 selectively induces chemotaxis in T helper type 2 cells, eosinophils, and basophils via seven-transmembrane receptor CRTH2. J. Exp. Med., 193, 255-261.
Hirawa et al. (2002) Lipocalin-type prostaglandin d synthase in essential hypertension. Hypertension, 39, 449-454.
Kagitani-Shimono et al. (2006) Lipocalin-type prostaglandin D synthase (beta-trace) is upregulated in the alphaB-crystallin-positive oligodendrocytes and astrocytes in the chronic multiple sclerosis. Neuropathol Appl Neurobiol, 32, 64-73.
Khanapure et al. (2007) Eicosanoids in inflammation: biosynthesis, pharmacology, and therapeutic frontiers. Curr Top Med Chem, 7, 311-340.
Li et al. (2004) Chronic behavioral testing after focal ischemia in the mouse: functional recovery and the effects of gender. Exp. Neurol., 187, 94-104.
Liu et al. (2007) Focal cerebral ischemia/reperfusion injury in mice induces hematopoietic prostaglandin D synthase in microglia and macrophages. Neuroscience, 145, 520-529.
Miwa et al. (2004) Identification of gene polymorphism in lipocalin-type prostaglandin D synthase and its association with carotid atherosclerosis in Japanese hypertensive patients. Biochem. Biophys. Res. Commun., 322, 428-433.
Mohri et al. (2003) Hematopoietic prostaglandin D synthase is expressed in microglia in the developing postnatal mouse brain. Glia, 42, 263-274.
Mohri et al. (2006a) Lipocalin-type prostaglandin D synthase is up-regulated in oligodendrocytes in lysosomal storage diseases and binds gangliosides. J. Neurochem., 97, 641-651.
Mohri et al. (2006b) Prostaglandin D2-mediated microglia/astrocyte interaction enhances astrogliosis and demyelination in twitcher. J. Neurosci., 26, 4383-4393.
Premkumar & Raisinghani (2006) Nociceptors in cardiovascular functions: complex interplay as a result of cyclooxygenase inhibition. Mol Pain, 2, 26.
Taniguchi et al. (2007) Prostaglandin D2 protects neonatal mouse brain from hypoxic ischemic injury. J Neurosci, 27, 4303-4312.
Tomimoto et al. (2002) A comparative study on the expression of cyclooxygenase and 5-lipoxygenase during cerebral ischemia in humans. Acta Neuropathol. (Berl), 104, 601-607.
Urade et al. (1993) Dominant expression of mRNA for prostaglandin D synthase in leptomeninges, choroid plexus, and oligodendrocytes of the adult rat brain. Proc. Natl. Acad. Sci. U.S.A., 90, 9070-9074.
Urade & Mohri (2006) [Sleep and brain function]. No To Hattatsu, 38, 331-333.
Weinberger, J. M. (2006) Evolving therapeutic approaches to treating acute ischemic stroke. J. Neurol. Sci., 249, 101-109.

1. A method for identifying an agent that may be useful in the treatment or prevention of ischemic brain injury, comprising:
contacting a lipocalin-type prostaglandin synthetase (L-PGDS) with a test compound;
determining whether the test compound increases enzymatic activity of the L-PGDS molecule; and
determining whether the test compound prevents or decreases a symptom of ischemic brain injury in an animal model.

2. The method of claim 1 wherein the L-PGDS molecule is in vitro.

3. The method of claim 1 wherein the L-PGDS molecule is in a cell.

4. A method for identifying an agent that may be useful in the treatment or prevention of ischemic brain injury, comprising:
contacting a mammalian cell which expresses L-PGDS with a test compound;
determining whether the test compound increases expression of the L-PGDS; and
determining whether the test compound prevents or decreases a symptom of ischemic brain injury in an animal model.

5. The method of claim 4 wherein the cell is a human cell.

6. The method of claim 4 wherein the cell is a leptomeningeal cell, a choroid plexus cell, or an oligodendrocyte.
